(19) United States
(12) Patent Application Publication
(10) Pub. No.: US 2018/0136140 A1
(43) Pub. Date: May 17, 2018

(54) SYSTEM FOR MONITORING AND MANAGING BIOMARKERS FOUND IN A BODILY FLUID VIA CLIENT DEVICE

(71) Applicants: Jon Brendsel, San Diego, CA (US); Shawn J. Green, Sacramento, CA (US)

(72) Inventors: Jon Brendsel, San Diego, CA (US); Shawn J. Green, Sacramento, CA (US)

(21) Appl. No.: 15/813,972

(22) Filed: Nov. 15, 2017

Related U.S. Application Data
(60) Provisional application No. 62/422,421, filed on Nov. 15, 2016.

(51) Int. Cl. G01N 21/78; G01N 33/52; G06F 19/00; G06T 7/90; H04N 1/60; A61B 5/00

(52) U.S. Cl. CPC G01N 21/78; G01N 33/521; G06F 19/322; G01N 2021/7759; H04N 1/6016; A61B 5/681; G06T 7/90

(57) ABSTRACT

Embodiments disclosed herein relate to a method monitoring a level of a biomarker found in a bodily fluid of a user. The client device receives an indication to capture a biomarker level reading of a test pad on a test strip. The test pad contains a reactant disposed thereon that, when placed into contact with a sample of the bodily fluid, displays a color related to a level of concentration of the biomarker in the bodily fluid. The client device identifies, with a camera, a portion of the test strip containing the test pad displaying the color related to the concentration level of the biomarker. The client device identifies the color displayed on the test strip. The client device correlates the identified color displayed on the test strip to the level of the biomarker. The client device updates the account of the user with the determined biomarker level.

### CROSS-REFERENCE TO RELATED APPLICATIONS

The present application claims priority to U.S. Application Ser. No. 62/422,421, filed Nov. 15, 2016, which is hereby incorporated by reference in its entirety. The present application is related to U.S. application Ser. No. 14/020,065, filed Sep. 6, 2013, the contents of which are incorporated by reference in its entirety.

### BACKGROUND

The present disclosure generally relates to a method and a system for self-monitoring, tracking, and correcting of lifestyle dietary patterns for maintaining wellness.

Americans spend billions of dollars annually on cardiovascular fitness, ranging from “healthy heart” diets rich in leafy greens to athletic shoes, to support vascular fitness. Last year alone, Americans spent over one billion dollars on a bagged salad brand that is known to be rich in natural cardioprotectives. An ever-growing number of Americans are willing to pay a premium to “keep in shape” and delay the onset of age-related diseases.

Cardiovascular disease (CVD) is the most expensive age-related disease that society manages. Around eighty one million Americans suffer from CVD. Americans spent five billion dollars last year alone to treat CVD. Three out of four aging Americans are aware of CVD and 90% of Americans would favor a daily dietary approach versus a prescription drug to sustain cardiovascular health.

While Americans typically believe prescription drugs should be covered by insurance, Americans appear to have no qualms about paying directly for dietary and lifestyle wellness strategies including self-monitoring devices, foods rich in cardioprotectives, and exercise. Self-diagnostics may seek to exploit wellness strategies where consumers have a high interest and willingness to pay out-of-pocket for self-administered, lifestyle-based strategies to increase vascular fitness and combat vascular ageing. The shift towards alternatives solutions is evidenced by the four hundred billion dollars spent on non-prescription based wellness strategies.

Furthermore, these alternative strategies to increase vascular wellness are highly social and valued. Online communities are becoming trusted resources for learning how to increase vascular wellness. For example, Pew research reported that about 61% of all adults obtain health information online, with this behavior growing exponentially. In similar fashion, there is also a growing demand to share personal wellness information with an open online wellness community, especially when compounded with the ability to increase performance and endurance abilities.

Recognizing that there is a growing list of natural whole foods rich in identifiable “bioactives” that enhance health and prevent disease, there continues to be a need for novel methods and devices that focus on vascular fitness and wellness, by allowing individuals to monitor their own health biomarkers and make dietary adjustments to sustain a healthy level. For example, leafy greens, such as arugula, beets, and spinach, among other vegetables, are rich with the precursor to Nitric Oxide, a potent natural cardio-protective and mediator that enhances stamina and endurance. What is needed is an easy to use and affordable device and method that enable users to monitor appropriate biomarker levels in conjunction with the ability to appropriately adjust dietary consumption of certain nutrients to improve health.

### SUMMARY

Embodiments disclosed herein generally relate to a method, system, and computer readable medium for monitoring a level of a biomarker found in a bodily fluid of a user using a client device. The client device receives an indication to capture a biomarker level reading. The biomarker level reading is of a test pad on a test strip. The test pad contains a reactant disposed thereon that, when placed into contact with a sample of the bodily fluid, displays a color related to a level of concentration of the biomarker in the bodily fluid. The client device identifies, with a camera, a portion of the test strip containing the test pad displaying the color related to the concentration level of the biomarker in the bodily fluid. The client device identifies the color displayed on the test strip. The client device correlates the identified color displayed on the test strip to the level of the biomarker. The client device updates the account of the user with the determined biomarker level.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrated only typical embodiments of this disclosure and are therefore not to be considered limiting of its scope, for the disclosure may admit to other equally effective embodiments.

FIG. 1 illustrates a computing environment, according to one embodiment.

FIG. 2 is a flow diagram illustrating a method of monitoring a level of a biomarker found in a bodily fluid, according to one embodiment.

FIG. 3 is a flow diagram illustrating a step of the method of FIG. 2 in more detail, according to one embodiment.

FIG. 4 illustrates a front view of the client device, according to one embodiment.

FIG. 5A is a flow diagram illustrating a step of the method of FIG. 2 in more detail, according to one embodiment.

FIG. 5B is a flow diagram illustrating a step of the method of FIG. 2 in more detail, according to one embodiment.

FIG. 6 is a block diagram of a computing platform, according to one embodiment.

FIG. 7 illustrates a computing environment, according to one embodiment.

FIG. 8 is a flow diagram illustrating a method of monitoring a level of a biomarker found in a bodily fluid, according to one embodiment.

FIG. 9 is a block diagram of a computing platform, according to one embodiment.

FIG. 10 is a flow diagram of a method of receiving health information from a user, according to one embodiment.

To facilitate understanding, identical reference numerals have been used, where possible, to designate identical elements that are common to the figures. It is contemplated that elements disclosed in one embodiment may be beneficially utilized on other embodiments without specific recitation.

### DETAILED DESCRIPTION

The present disclosure generally relates to a method and system for self-monitoring, tracking, and correcting lifestyle dietary patterns for maintaining wellness. For example, the present disclosure relates to leveraging a test strip (for bodily fluids such as saliva or urine) that detects wellness factors or metabolites, which are reflective of health, and recording/tracking the factors in context of a lifestyle adjustment, such as exercise or diet. More specifically, the system may be used as part of a behavioral modification program for dietary control, heart healthy food consumption, or general fitness. In particular, the present disclosure, according to one embodiment, relates to an apparatus used in conjunction with a software platform for monitoring Nitric Oxide (NO) rich foods consumption and/or cardiovascular protection of an individual. As will be made evident by the following discussion, the apparatus and software platform may be used to monitor any biomarker, including but not limited to, Nitric Oxide, uric acid, ketones, and the like.

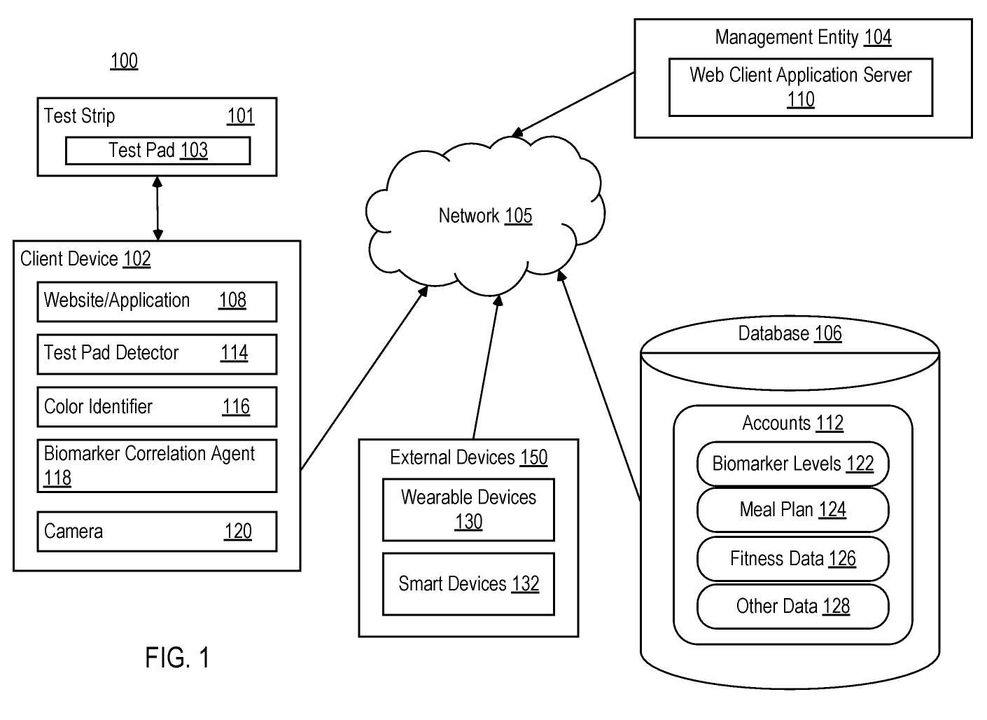

FIG. 1 illustrates a computing environment 100, according to one embodiment. The computing environment includes a client device 102, a management entity 104, and a database 106. The client device 102 is configured to capture information associated with a test strip 101 having a test pad 103 thereon. The test strip 101 contains a scored mark (or crease) at the midpoint of the strip 101 and wherein the strip 101 contains an absorbent pad at each end. The scored mark enables the strip 101 to be folded easily, thereby, allowing pads at each end of the strip 101 to make contact. The strip 101 contains a first absorbent pad at one end and a second absorbent pad at the opposite end: the first absorbent pad comprises a fluid collection pad, and the second absorbent pad comprises test pad 103. The fluid collection pad may comprise a wicking pad, membrane, paper, resin, sponge, immunoabsorbent pad, ionic or other suitable platform that absorbs saliva analytes to be transfer to the test reagent pad, known to those skilled in the art. The test pad 103 enables dry reagent detection chemistry comprising components modified from the Griess diazotization reaction, comprising mixture of naphthylenediamine-dihydrochloride, and sulphanilamide in acidic solution or para-arsanilic acid; and other reactive components known to those skilled in the art. In certain embodiments, the test pad 103 comprises more than one testing zone so that the fluid may be analyzed for more than one biomarker.

When the dry chemical reagents come into contact with the bodily fluid containing wellness factors or metabolites, a color product is displayed. The intensity of the color product is correlated to a concentration of wellness factors or metabolites found in the bodily fluid.

The client device 102 is configured to capture the a portion of the test strip 101, comprising the test pad 103, that has changed colors to indicate a biomarker level. The client device 102 may be any type of computing device accessible by a user, such as, but not limited to, a computer, a mobile device, a tablet, and the like. Generally, client device 102 may include components of a computing device, e.g., a processor, memory, hard disk drive, input/output device, and the like. As illustrated, the client device 102 includes a web client (or application) 108.

The web client 108 allows a user of the client device 102 to access a functionality of the management entity 104. For example, web client 102 may access a nutrition platform, such as Berkeley Fit® connected nutrition platform, commercially available from Berkeley Fit, LLC.

In the embodiments described below, a user operating client device 102 may communicate over network 105 to request access to an application or webpage from web client application server 110. For example, client device 102 may be configured to execute web client 108 to access content managed by web client application server 110. The content that is displayed to a user may be transmitted from web client application server 110 to the client device 102, and subsequently processed by web client 108 for display through a graphical user interface (GUI) of the user's client device 102.

In one example, a webpage displayed on the client device 102 is the user's personal webpage on the Berkeley Fit® connected nutrition platform. The management entity 104 is in communication with database 106. For example, the management entity may communicate with database 106 via a local connection (e.g., storage area network (SAN), network attached storage (NAS), or over the internet (i.e., cloud based storage service). The management entity 104 is configured to either directly access data included in database 106 or interface with a database manager (not shown) that is configured to manage data included within the database 106.

User of client device 102 may be associated with one or more accounts 112 stored in database 106. The account 110 is a data object that stores data associated with the user. For example, the account 110 may include information such as the user's email address, password, contact information, and the like. As illustrated, each account 112 includes at least biomarker recordings 122, meal recordings 124, exercise recordings 126, and one or more other health recordings 128.

The biomarker recordings 122 may include biomarker levels measured by the test strip 101 over a period of time. For example, biomarker recordings 122 may include measured biomarker levels organized by date, such that a user may view a snapshot of the user's biomarker levels at any given time. The meal recordings 124 may include an entry listing each item the user has eaten over a period of time. For example, in a given day, a user may enter each item of each meal the user has eaten. This information may include the name and amount of the food. In one embodiment, the platform may be configured to calculate the nutrition information of the meal based off the name of the food and the amount provided. The exercise recordings 126 may include an entry listing the type of exercise and the duration of exercise performed by the user over a period of time. For example, the user may enter that the user swam for forty-five minutes on Monday. The various health recordings 128 may include one or more recordings related to sleep, blood pressure, heart rate, and the like over a period of time. For example, other health related information 128 may include, but is not limited to, patterns of physiological data, patterns of contextual data, patterns of activity data derived from detected/recorded information, and the like.

The test strip 101 in conjunction with the client device 102 aids in allowing the user to track personal wellness trends, thereby allowing health planning, dietary intervention, and reporting capability to sustain or improve health. As recited above, the client device 102 allows a user to manually enter information directed to biomarker levels, meal recordings, exercise recording, and various measurement recordings related to the health of the user. In another embodiment, the client device 102 allows to user to record biomarker levels by leveraging a camera of the client device 102.

The client device 102 further includes a test pad detector 114, a color identifier 116, a biomarker level correlation agent 118, and a camera 120. The camera 120 is configured to capture the test pad 103 using the client device 102. In some embodiments, one or more of test pad detector 114, color identifier 116, and biomarker level correlation agent 118 may reside on management entity 114. In one embodiment, the camera 120 may be configured to automatically identify the test pad 103 through use of test pad detector 114. In another embodiment, the camera 120 may be configured to capture the test pad 103 when prompted by the user. The color identifier 116 is configured to detect the activation color indicated by the test pad 103 in the captured image. The biomarker level correlation agent 118 correlates the identified activation color with a biomarker level. Together, the camera 120, the test pad detector 114, the color identifier 116, and the biomarker level correlation agent 118 aid in monitoring and capturing biomarker levels of the user.

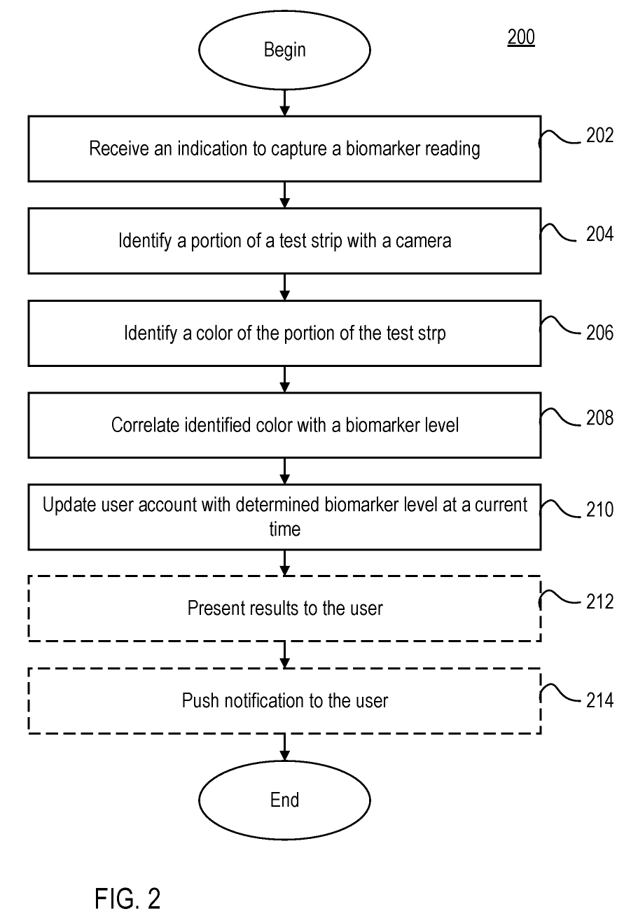

FIG. 2 is a flow diagram illustrating a method 200 of monitoring a level of a biomarker found in a bodily fluid, according to one embodiment. As previously discussed, the test strip 101 may be used to measure a level of a biomarker that includes, but not limited to, NO, uric acid, and ketones. The test strip 101 measures the level of the biomarker using a sample of bodily fluid (e.g., saliva or urine), and applies an activation agent to the sample, thereby producing a reaction that turns the sample a color that corresponds to a biomarker level.

For example, the test strip 101 may include an absorption pad and test pad 103. The absorption pad may be on a first end of the test strip 101 and the test pad 103 may be on a second end of the test strip 101. The test pad 103 and absorption pad being positioned on the same face of the test strip 101. The absorption pad is configured to receive a bodily fluid for testing. In the specific example of saliva, the first end of the test strip is inserted under the tongue, or sublingually, for a period of time (e.g., 3-10 seconds) to absorb saliva. The user then folds the test strip such that the absorption pad and the test pad 103 make physical contact. For example, the test pad 103 includes a reactant that, when placed into contact with the bodily fluid of the absorption pad, displays a color indicative of a biomarker level. For example, the absorption pad and the test pad 103 may make contact for about 3-5 seconds. Upon release and separation of the absorption pad from the test pad 103, a colorimetric reaction, based on the chemical detection reagents used, will take place (e.g., within about 10-60 seconds) on the test pad 103, resulting in a color intensity and hue that correlates with a concentration of the biomarker.

In one embodiment, in which the test strip 101 is used to measure NO levels, the test pad 103 of the test strip 101 provides a concentration range of the NO metabolite from about 25 to greater than 400 umol/L nitrite with visibly distinct colorimetric sub-ranges corresponding to (umol/L, ppm, mg/L), specifically, but not limited to: 0 to 25, 25 to 100, 100 to 200, 200 to 350, and greater than 400 umol/L nitrite.

The method 200 provides a method of monitoring the biomarker levels by capturing the test pad 103 with the client device and subsequent processing. The method 200 begins at step 202. At step 202, the client device 102 receives an indication that the user desires to capture a biomarker reading. For example, the client device 102 may access the web client 108 and navigate to a Biomarker Recording page that prompts the user to identify the test pad 103 using the client device 102. To capture the test pad 103, the user must place the test pad 103 in a line of sight of the camera 120.

At step 204, the client device identifies a portion of the test strip 101 containing the test pad 103 using the camera 120. In one embodiment, the client identifies the test pad 103 using the camera 120 by physically pressing a button on the client device or a portion of screen of the client device (e.g., touch screen device) to save and transmit an image of the test pad 103 to the management entity 104 for processing. In another embodiment, the camera 120 and the test pad detector 114 work in conjunction to identify the test pad 103. The client device 102 does not need to save an actual image of the test strip 101. Rather, analysis may be performed on the test pad 103 in real time while the test strip is being identified by the camera 120.

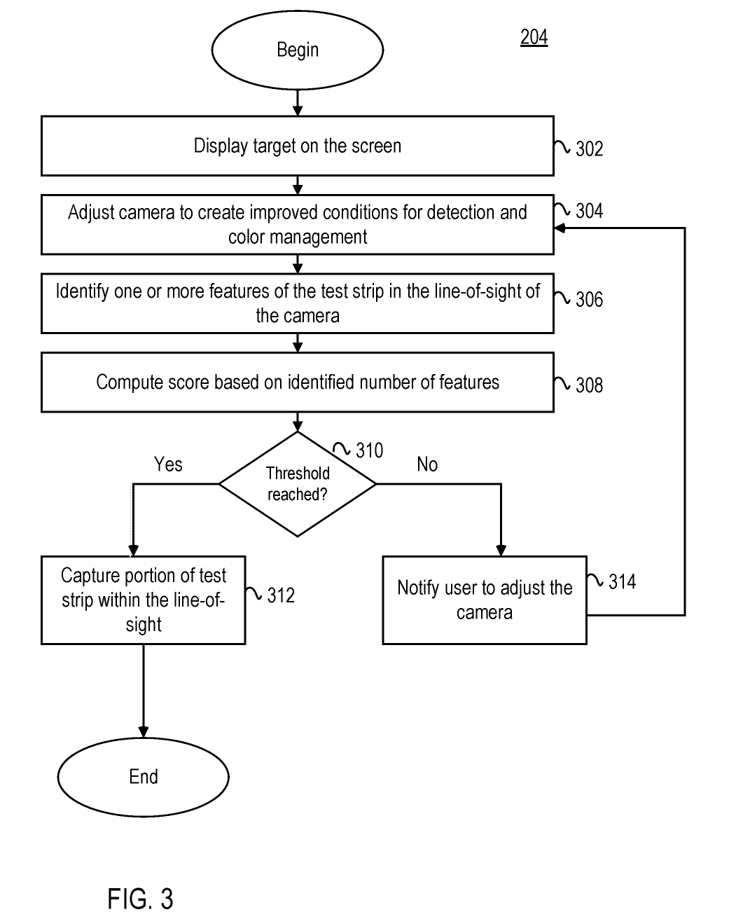

FIG. 3 is a flow diagram illustrating step 204 of method 200 in more detail, according to one embodiment. For example, the steps discussed in FIG. 3 are directed to the embodiment in which the camera 120 and the test pad detector 114 work in conjunction in identifying the test pad 103, without a prompt (e.g., physical, audible, etc.) of the client device 102 by the user.

At step 302, the client device 102 displays a target on the screen. The target helps guide the user in positioning the test pad 103 within the bounds defined by the target. In one example, when a portion of the test strip (i.e., the test pad 103 portion undergoing the color change indicating a biomarker level) is within the bounds defined by the target, the client device 102 automatically identifies the test strip 101.

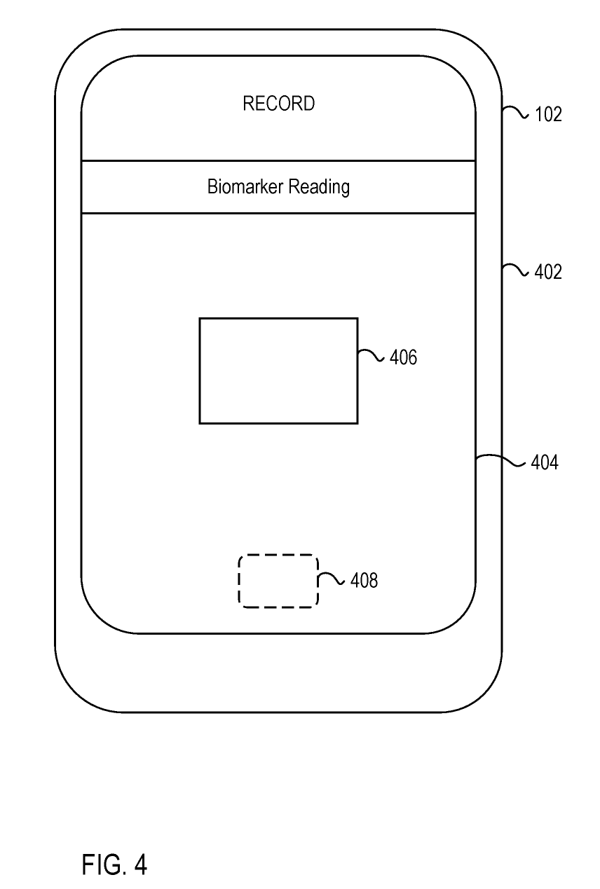

FIG. 4 illustrates a front view of the client device 102, according to one embodiment. The client device 102 includes a first side 402. The first side 402 includes a screen 404 displaying a GUI 406 of the web client 108. For example, the GUI 406 shown illustrates a “Record” screen, in which the camera 120 is activated. When the camera 120 is activated, the GUI 406 is updated to display everything within the line of sight of the camera 120. Thus, if, for example, the user is attempting to capture the test strip 101 lying flat on a desk, the GUI 406 would be updated to display any portions of the desk and the test strip 101 that are within the line of sight of the camera. The GUI 406 further includes a target 404 (e.g., rectangular target, crosshair, or the like) superimposed over the image currently being captured by the camera 120. In some embodiments, an exposure button 408 may also be superimposed over the image. The exposure button 408 may be used to bypass the automatic identification of the strip 101.

Referring back to FIG. 3, after the target 404 is displayed on the screen of client device 102, the user of client device 102 may move the client device 102 relative to the test strip 101 (or the test strip 101 relative to the client device 102) in an attempt to trigger automatic capture by the camera 120. At step 304, the test pad detector 114 manipulates the camera 120 to create improved conditions for detection and color measurement. For example, the test pad detector 114 may manipulate the camera 120 by adjusting white balance and exposure (or brightness) automatically. To do this, the test pad detector 114 focuses the camera on the test pad 103 of the test strip. The camera 120 may then take a weighted average of the picture to determine an improved exposure and white balance level.

At step 306, once the improved conditions of the camera 120 are set, the test pad detector 114 identifies one or more features of the test strip 101 within the line of sight of the camera 120. For example, the test pad detector 114 may search for the presence of a black line within a certain distance of the target 404. In another example, the test pad detector 114 may search for a concentration of white color of the test strip 101, i.e. plain areas of the test strip 101. In another example, the test pad detector 114 may search for a concentration of certain colors that are known to be present on the test pad 103. In another example, the test pad detector 114 may search for edges of the tests strip 101 and edges of the test pad 103 with respect to the target 404. In another example, the test pad detector 114 may search for the present of boundaries of colors, i.e. between the test pad 103 and the surrounding white paper of the test strip 101.

At step 308, the test pad detector 114 computes a score based on one or more of the identified features of the test strip 101 in step 306. The computed score reflects an overall confidence that a sufficient number of features exist to conclude that what is in the line of sight of the camera 120 is the test strip 101. The test pad detector 114 then compares computed score to a threshold score. The threshold score indicates a minimum allowed confidence to identify a test strip.

If at step 310, the test pad detector 114 determines that the computed score is greater than or equal to the threshold score, then at step 310, the test pad detector 114 instructs the camera 120 to capture whatever is displayed within the target 404. If, however, at step 310, the test pad detector 114 determines that the computed score is less than the threshold score, then the test pad detector 114 provides feedback to the user, instructing the user to adjust the camera 120 (step 314). The method then proceeds back to step 304.

Referring back to FIG. 2, after the client device 102 captures an image of the test pad 103 with the camera 120, at step 204 the client device 102 identifies a color of the test pad in the captured image. The color of the test pad is indicative of the level of the biomarker detected.

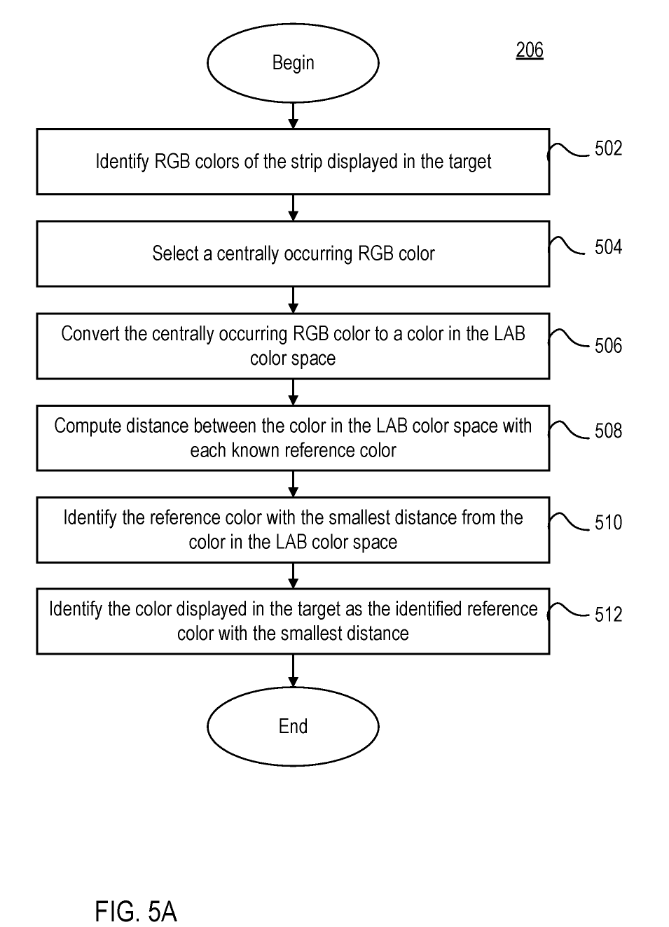

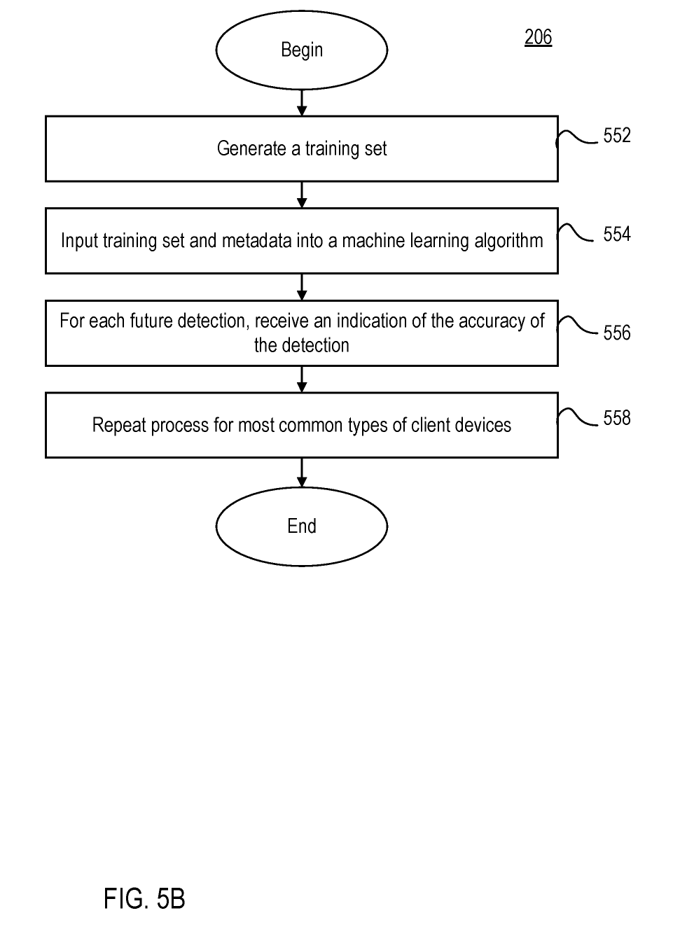

FIG. 5A is a flow diagram illustrating step 204 of method 200 in more detail, according to one embodiment. At step 502, the color identifier 116 identifies red-green-blue (RGB) colors of the test pad 103 on the test strip 101. For example, the color identifier 116 works with the processor of the client device 102 to differentiate between one or more colors using values for red, green, and blue, respectively.

At step 504, the client device 102 identifies the most frequent color on the test pad 103. For example, the color identifier 116 may identify the test pad 103 as containing more than a single color during the RGB detection. In this scenario, the color identifier 116 determines a centrally occurring color. In other words, client device 102 generates a histogram for all colors identified on the test pad 103. Color identifier 116 may then partition the histogram into quintiles, and remove the top two quintiles and the bottom two quintiles. The color identifier may then determine an average color of the remaining quintile.

At step 506, the color identifier 116 converts the identified RGB color of the test pad 103 to the LAB color space. The LAB color space mathematically describes all perceivable colors in the three dimensions: L for lightness and a and b for the color opponents red-green and blue-yellow. Unlike the RGB color model, the LAB color space is designed to mimic human vision. Thus, converting the RGB color to the LAB color spaces provides a more accurate reading of the color displayed by the test pad 103.

At step 508, the color identifier 116 computes a distance, in the LAB color space, between the color of the test pad 103 and each reference color provided by the test strip manufacturer. In one specific example, in the LAB color space, there is an algorithm referred to as International Commission on Illumination (CIE) 2000 (i.e., CIE2000). The CIE2000 algorithm computes the distance between any two colors in the LAB color space, while accounting for factors, such as, hue rotation, neutral colors, lightness, chroma, and hue. In other examples, sufficient algorithms may include CIE76, CIE94, and the like. Generally, any algorithm that takes into account a Euclidean distance between any two colors in the LAB color space may be used. The algorithms are used to compute the distance between the detected color and the known reference colors provided by the strip manufacturer.

At step 510, the color identifier 116 identifies reference color with the minimum computed distance from the detected color. For example, at step 508, the color identifier 116 computed the distance between the detected color and each reference color provided by the strip manufacturer. In one embodiment, there are nine reference colors, which correspond to nine computed distances between each of the nine references colors and the detected color. The color identifier 116 then identifies the reference color for which the distance between it and the detected color is the smallest. The color identifier 116 then identifies the detected color as the reference color with the smallest computed distance (step 512).

The steps discussed above in conjunction with FIG. 5 are not limited to particular color spaces. For example, one of ordinary skill in the art can imagine a color identifier 116 identifying a color of the test pad 103 using a color space that is not the RGB color space. In another example, one or ordinary skill in the art can imagine color identifier 116 generally converting a color from a first generic color space to a second generic color space. The use of RGB and LAB color spaces is merely one embodiment for identifying the color of the test pad 103.

FIG. 5B is a flow diagram illustrating step 204 of method 200 in more detail, according to another embodiment. The method discussed below in conjunction with FIG. 5B differs from that in FIG. 5 in that, rather than relying on a Euclidean distance algorithm to measure a best color match as discussed above in FIG. 5A, the present embodiment leverages the camera 120 to accurately measure the color of the test pad 103. For example, the steps discussed below make use of machine-learning techniques to train the algorithm on a per device basis that leverages crowdsourcing techniques.

At step 552, client device 102 generates a training set. A user of client device 102 begins with a stock solution (e.g., sodium nitrite when testing for NO levels) of a known concentration. The user would generate a series of dilutions of this stock solution to create additional solutions of known quantities. Each reference solution is used to expose test strips 101. To generate the training set, client device 102 captures a series of photos of the exposed test strips.

At step 554, the client device 102 inputs the captured series of photos into a machine learning algorithm. In one example, the machine learning algorithm is hosted on management entity 104. Thus, in this example, client device 102 would transmit the captured series of photos to management entity 104. In some embodiments, along with the captured series of photos, client device 102 inputs metadata associated with camera 120. Such metadata may include, for example, shutter speed, color balance, International Standards Organization (ISO) sensitivity, and the like. The machine learning algorithm employed is client device specific. For example, for all users of a client device of type A, a machine learning algorithm of type A may be used, while for all users of a client device of type B, a machine learning algorithm of type B may be used.

At step 556, for each future detection of a test pad, a user of client device 102 may be asked to confirm whether the result generated was deemed accurate. If the result was deemed to be inaccurate, the user of the client device would input the corrected value for the test pad 103. Such results are fed back into the training set for that algorithm. As this step may be performed by each client device using the platform, the current method effectively crowdsources all devices of a certain type to more accurately identify a color of a test pad in subsequent recordings.

At step 558, the entire process may be repeated for the “most common” devices. While the above method primarily involved common client devices, for other less common devices, a generic algorithm may be used. In this scenario, the crowdsourcing performed in step 556 plays a larger role in generating correct calibrations for that particular device type.

Referring back to FIG. 2, after the color of the test pad 103 is identified, the client device correlates the detected color with a biomarker level. As referred to above, the strip manufacturer provides one or more reference colors. Each reference color corresponds to a specific biomarker level, or range of biomarker levels. In one embodiment, the biomarker levels may be stored in database 106. As such, when correlating the detected color with its respective biomarker level, the biomarker level correlator 118 may refer to the biomarker levels stored in database 106. In one example, the biomarker level correlator 118 requests access to a portion of the database 106 (i.e., the portion of which stores the biomarker levels and reference colors) to determine the biomarker level of the detected color. In another example, the biomarker level correlator 118 does not have direct access to the database 106. Rather, the biomarker level correlator 118 communicates with the management entity 104 in determining the biomarker level of the detected color. As such, the biomarker level correlator 118 transmits the detected color to the management entity 104, which subsequently accesses the database 106 to identify the biomarker level associated with the reference color. The management entity 104 may optionally transmits the corresponding biomarker level back to the biomarker level correlator 118.

At step 210, the client device 102 updates the user's account with the determined biomarker level. For example, in one embodiment, the user may instruct the client device 102 to update the user's account with the determined biomarker level at the time the client device 102 determine the biomarker level. In another example, the client device 102 may automatically update the user's account with the determined biomarker level as part of the method 200. In both scenarios, the client device 102 may communicate with the management entity 104 to gain access to the user's account 112 either directly (directly uploading the information) or indirectly (transmitting information to the management entity for upload).

In some embodiments, the method 200 may include steps 212 and 214. At step 212, the client device 102 receives a message from the web client application server 110. The web client application server 110 may generate such a push notification for a variety of reasons. For example, the web client application server 110 may generate a push notification that notifies the user the biomarker level that was recorded exceeds a threshold amount. In another example, web client application server 110 may generate a push notification that notifies the user the biomarker level that was recorded puts the user on a pace that falls short of the user's target biomarker level. In a specific example, the web client application server 110 may generate a corrective course of action that enhances wellness and fitness by changing a user's exercise or diet play to aid in elevating biomarker levels of the user. The client device 102 subsequently updates the GUI to display the received push notification (step 214).

In some embodiments, the method 200 may include one or more information display steps. For example, when analyzing the recorded biomarker level, the web client application server 110 may take into account other health information associated with the user. For example, the web client application server 110 may access meal recordings 124, exercise recordings 126, and the like to analyze the recorded biomarker level in the context of the additional health information. In a specific example, the web client application server 110 may specifically take into account the intensity and duration of the user's exercise recordings for the day when analyzing the recorded biomarker levels. In another specific example, the web client application server 110 may specifically take into account an NO potency of the foods recorded by the user when evaluating a recorded NO level.

The web client application server 110 may then generate a graphical representation of the biomarker status in the context of time after exercise and/or consumption of food on a daily, weekly, monthly basis. This graph is then transmitted to the user device 102, which subsequently updates the GUI to display the graphical representation. The user may subsequently share this report (or graph) on various social media platforms.

In another embodiment, the health information output may be used by another device. For example, a person skilled in the art could imagine a scenario in which a smart device or wearable gains access to the outputted health information and subsequently leverages that information in one or more applications.

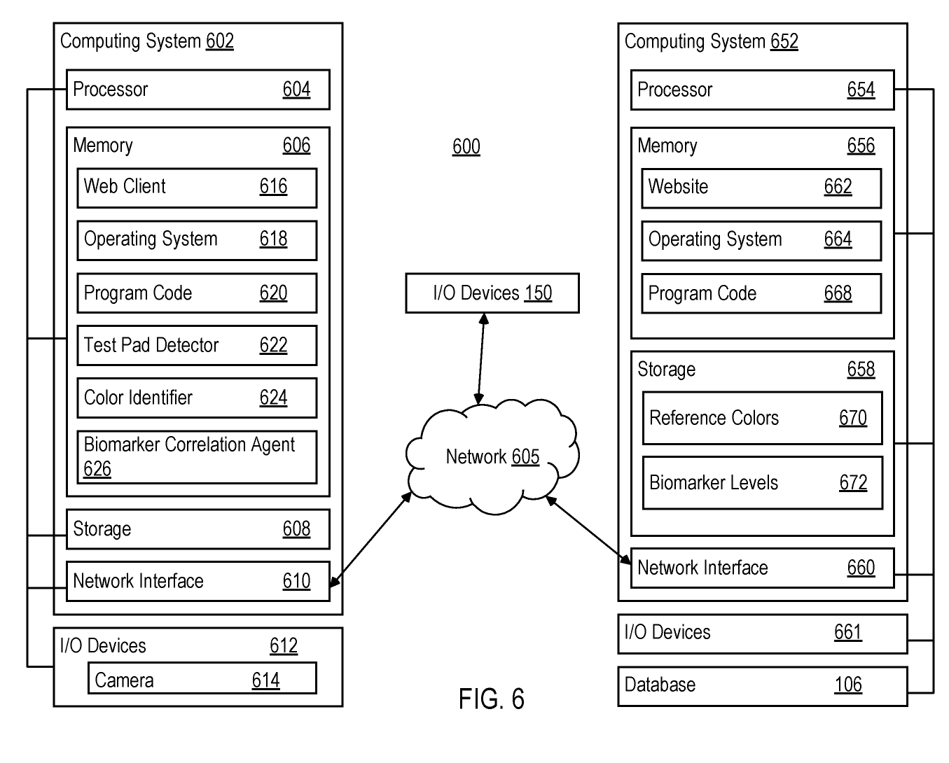

FIG. 6 is a block diagram of a computing platform 600, according to one embodiment. The computing platform 600 includes a computing system 602 (e.g., client device 102) and a computing system 604 (e.g., web client application server 110) communicating over network 605.

The computing system 602 includes a processor 604, a memory 606, storage 608, and a network interface 610. The computing system 602 may be coupled to one or more I/O devices 612. The one or more I/O devices 602 include a camera 614. The camera 614 is configured to identify and/or capture anything within its line of sight (e.g., a test strip 101).

The processor 604 retrieves and executes programming instructions 620 stored in memory 606, as well as stores and retrieves application data. The processor 604 is included to be representative of a single processor, multiple processors, a single processor having multiple processing cores, and the like. The storage 608 may be a disk drive storage device. Although shown as a single unit, the storage 608 may be a combination of a fixed and/or removable storage devices, such as fixed disk drives, removable memory cards, optical storage, network attached storage (NAS), or storage area network (SAN). The network interface 610 may be any type of network communications allowing the computing system 602 to communicate with computing system 650 via network 605. Furthermore, as will be understood by one of ordinary skill in the art, any computer system capable of performing the functions described herein may be used.

In the embodiment, the memory 606 includes web client 616, operating system 618, program code 620, test pad detector 622, color identifier 624, and biomarker level detector 626. The web client 616 is configured to access webpages and/or content managed by computing system 650. For example, the web client 616 may access the user's personal page on the Berkeley Fit® connected nutrition platform. The program code 620 may be accessed by the processor 604 for processing. The program code 620 may include the steps discussed herein in conjunction with FIGS. 2, 3, 5, and 10, that are performed by the client device. The test pad detector 622 is configured to allow the computing system 602 to identify the test pad 103 automatically through use of the camera 614. The color identifier 624 is configured to detect the color that is indicated by the test pad 103 in the captured image. The biomarker level correlation agent 626 is configured to correlate the identified activation color with the biomarker level. Together, the camera 614, the test pad detector 622, the color identifier 624, and the biomarker level correlation agent 626 aid in monitoring and capturing biomarker levels of the user.

Although memory 606 is shown as a single entity, memory 606 may include one or more memory devices having blocks of memory associated with physical addresses, such as random access memory (RAM), read only memory (ROM), flash memory, or other types of volatile and/or non-volatile memory.

The computing system 652 includes a processor 654, a memory 656, a storage 658, and a network interface 660. The computing system 652 may be coupled to one or more I/O devices 661.

The processor 654 retrieves and executes programming instructions 664 stored in memory 656, as well as stores and retrieves application data. The processor 654 is included to be representative of a single processor, multiple processors, a single processor having multiple processing cores, and the like. The network interface 660 may be any type of network communications allowing the computing system 652 to communicate with computing system 602 via network 605. The storage may 658 may be a disk drive storage device. Although shown as a single unit, the storage 658 may be a combination of a fixed and/or removable storage devices, such as fixed disk drives, removable memory cards, optical storage, network attached storage (NAS), or storage area network (SAN). As illustrated, the storage 658 may include reference colors 670 and biomarker levels 672. The reference colors 670 may include those reference colors that are provided by the manufacturer to which the computing environment 600 will compare the color of the captured image. For example, the reference colors 670 may include a separate color directed to “depleted” (poor biomarker level), then “low”, then “threshold” (adequate biomarker level), then “target” and finally “high”. The biomarker levels 672 include those readings to which each color represents. For example, the biomarker levels 672 include the correlations between a single color and a single level.

In the embodiment, the memory 656 includes an operating system 662, program code 664, and website 668. The website 668 is accessed by the computing system 602. The website 668 may correspond to a user's personal webpage that is managed by the web client application server. The program code 664 may be accessed by the processor 664 for processing. The program code 662 may include the steps discussed herein in conjunction with FIGS. 2, 3, 5, and 10, that are performed by the web client application server.

Although memory 656 is shown as a single entity, memory 656 may include one or more memory devices having blocks of memory associated with physical addresses, such as random access memory (RAM), read only memory (ROM), flash memory, or other types of volatile and/or non-volatile memory.

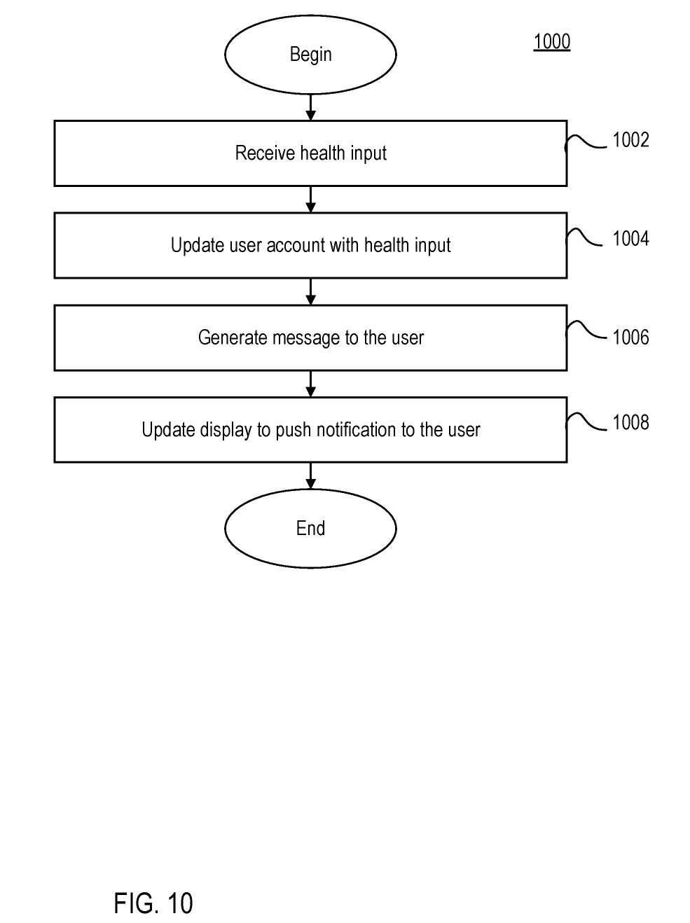

The computing environment 600 may further include one or more external devices 150. The one or more external devices 150 are configured to measure and record a health metric associated with the user. For example, the one or more external devices 150 may include wearable device(s) 152 and smart device(s) 154. An example wearable device 152 may include a smart watch that may be configured to track daily steps, calories burned, heart rate, sleep cycle, and the like. An example smart device 154 may be a wireless blood pressure device, a wireless weight scale, a wireless body fat monitor, and the like. The one or more external device 150 are discussed in more detail below in conjunction with FIG. 10.

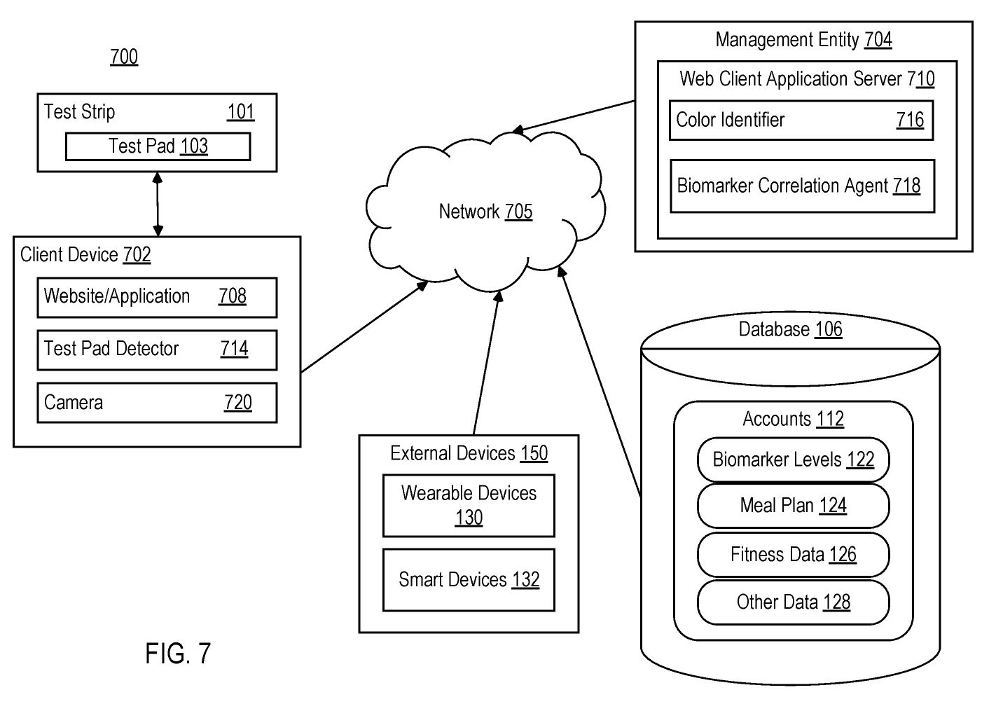

FIG. 7 illustrates a computing environment 700, according to one embodiment. The computing environment includes a client device 702, a management entity 704, and the database 106. The computing environment 700 is substantially similar to computing environment 100. For example, the computing environment 700 includes similar components as those illustrated in computing environment 100. However, rather than the client device 702 including the color identifier and the biomarker level correlator, the client device 702 includes a website/application 708 (substantially similar to website/application 108) and a camera 720 (substantially similar to camera 120). In some embodiments, the client device 702 may also include a test pad detector 714 (substantially similar to test pad detector 114). In other embodiments, test pad detector 714 may reside in management entity 704. The management entity 704 includes a web client application server 710. The web client application server 710 is substantially similar to web client application server 110. The web client application server 710 further includes a color identifier 716 and a biomarker level correlator 718.

In operation, the computing environment 700 differs from the computing environment 100 in that the user of the client device 702 takes a picture of the test pad portion of the test strip 101 and transmits (or uploads) that picture to the web client application server 710 for further analysis. For example, the web client application server 710 includes a color identifier 716 (substantially similar to color identifier 116) and the biomarker level correlator 718 (substantially similar to biomarker level correlator 118). The color identifier 716 is configured to identify the color of the test pad 103 of the test strip 101 after the web client application server 710 receives the image of the test pad 103 from the client device 702. The color identifier 716 identifies the color of the test pad 103 using a method similar to that of method 300. The biomarker level correlator 718 is configured to correlate the identified color of the test pad with a biomarker level. For example, the biomarker level correlator 718 is configured to correlate the identified color with a biomarker level.

Additionally, similar to management entity 104, the management entity 704 is further in communication with database 106. For example, management entity 704 is configured to access one or more accounts 112 of the user to update biomarker level information, exercise information, meal plan information, and the like.

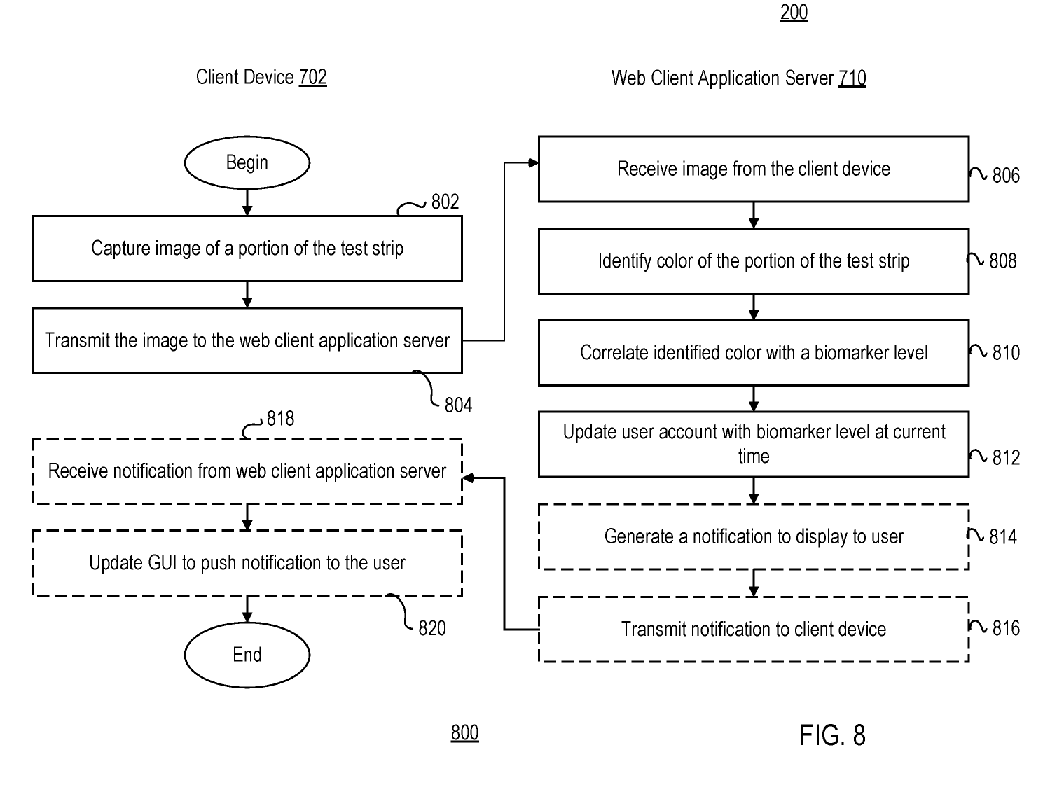

FIG. 8 is flow diagram of a method 800 of monitoring a level of a biomarker found in a bodily fluid, according to one embodiment. The method 800 is implemented in computing environment 700, although one of ordinary skill in the art could imagine the method 800 being implemented on other computing environments.

The method 800 begins at step 802. At step 802, the client device 702 captures an image of the test strip 101 using the camera 120. For example, in one embodiment, the client device 702 may receive an indication from the user to capture an image of a portion of the test strip 101 that is in the line-of-sight of the camera 120. In another embodiment, the client device 702 may leverage the test pad detector 714 to automatically capture an image of the test pad 103 portion of the test strip 101 that is in the line-of-sight of the target 404 portion of the camera 120.

At step 804, the client device 702 transmits the captured image to the web client application server 710 over network 705. At step 806, the web client application server 710 receives the image from the client device 702. The web client application server 710 continues to analyze the image of the test pad 103 for biomarker levels.

At step 808, the web client application server 710 identifies the color of the test pad 103. For example, the web client application server 710 identifies the color of the test pad on the test strip 101, using the method discussed above in conjunction with FIG. 3. Rather than the client device 102 performing the actions in FIG. 3, in the present embodiment, the web client application server 710 performs the steps of identified RGB colors of the test pad 103 of the test strip 101, identifying the most frequent RGB color of the test pad, converting the identified RGB color to a LAB color space, computing the distances, in the LAB color space, between the detected color of the test pad 103 and that of each reference color, identifying the reference color with the minimum computed distance from the detected color, and identifying the detected color as the reference color with the smallest computed distance.

At step 810, the web client application server 710 correlates the identified color with a biomarker level. As referred to above, the strip manufacturer provides one or more reference colors. Each reference color corresponds to a specific biomarker level, or range of biomarker levels. In one embodiment, the biomarker levels may be stored in database 106. As such, when correlating the detected color with its respective biomarker level, the biomarker level correlator 718 may refer to the biomarker levels stored in database 106. The biomarker level correlator 718 accesses the database 106 to identify the biomarker level associated with the reference color.

At step 812, the web client application server 710 updates the user's account with the determined biomarker level. For example, in one embodiment, the client application server 710 includes temporal information along with the biomarker level recording. Such temporal information provides a more complete picture to the user's biomarker level history.

In some embodiments, method 800 may further include steps 814-820. At step 814, the web client application server 710 may generate a notification in response to the user's biomarker level recording. For example, the web client application server 710 may determine that the user has reached a sufficient biomarker level for the day, and notify the user as such. In another example, the web client application server 710 may determine that the user is on pace to fall short of a desired biomarker level, and notify the user as such. At step 816, the web client application server 710 transmits the generated message to the client device 702 over network 705.

At step 818, the client device 702 receives the generated notification from the web client application server 710. The client device 702 pushes the notification to the user (step 820). For example, the client device 702 may update GUI displayed on the screen to display the notification message to the user.

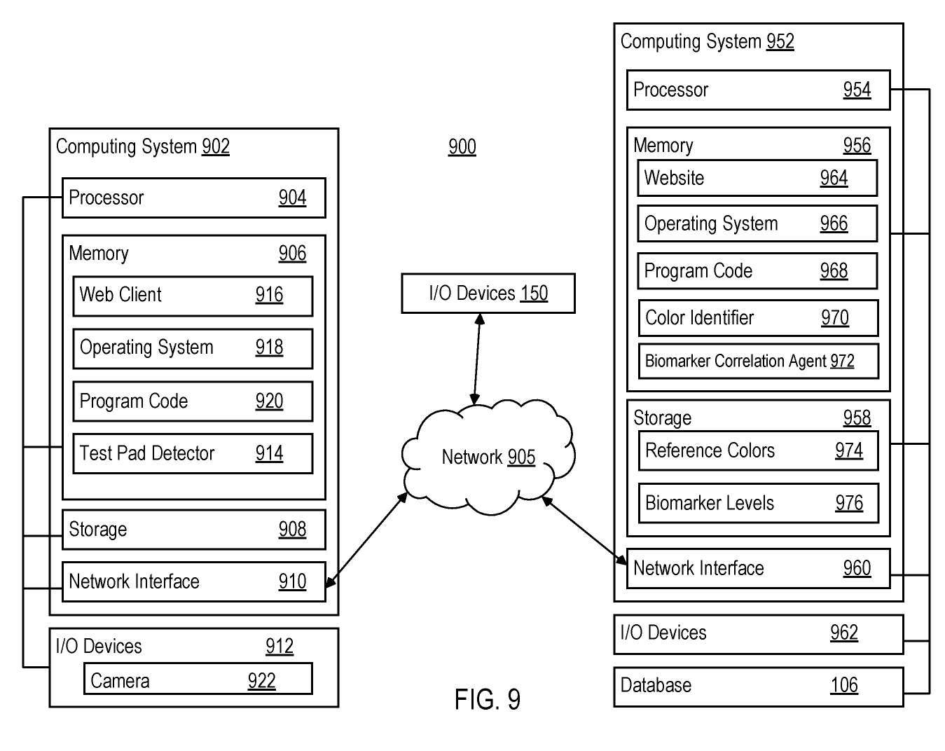

FIG. 9 is a block diagram of a computing platform 900, according to one embodiment. The computing platform 900 includes a computing system 902 (e.g., client device 702) and a computing system 904 (e.g., web client application server 710) communicating over network 905.

The computing system 902 includes a processor 904, a memory 906, storage 908, and a network interface 910. The computing system 902 may be coupled to one or more I/O devices 912. The one or more I/O devices 912 include a camera 922. The camera 922 is configured to capture anything within its line of sight (e.g., a test strip 101).

The processor 904 retrieves and executes programming instructions 920 stored in memory 906, as well as stores and retrieves application data. The processor 904 is included to be representative of a single processor, multiple processors, a single processor having multiple processing cores, and the like. The storage 908 may be a disk drive storage device. Although shown as a single unit, the storage 908 may be a combination of a fixed and/or removable storage devices, such as fixed disk drives, removable memory cards, optical storage, network attached storage (NAS), or storage area network (SAN). The network interface 910 may be any type of network communications allowing the computing system 902 to communicate with computing system 952 via network 905. Furthermore, as will be understood by one of ordinary skill in the art, any computer system capable of performing the functions described herein may be used.

In the embodiment, the memory 906 includes test pad detector 914, web client 916, operating system 918, and program code 920. The web client 916 is configured to access webpages and/or content managed by computing system 952. For example, the web client 916 may access the user's personal page on the Berkeley Fit® connected nutrition platform. The program code 920 may be accessed by the processor 904 for processing. The program code 920 may include the steps discussed herein in conjunction with FIGS. 3, 5, 8, and 10 that are performed by the client device. The test pad detector 914 is configured to allow the computing system 902 to capture the test pad 103 automatically through use of the camera 922.

Although memory 906 is shown as a single entity, memory 906 may include one or more memory devices having blocks of memory associated with physical addresses, such as random access memory (RAM), read only memory (ROM), flash memory, or other types of volatile and/or non-volatile memory.

The computing system 952 includes a processor 954, a memory 956, a storage 958, and a network interface 660. The computing system 952 may be coupled to one or more I/O devices 962.

The processor 954 retrieves and executes programming instructions 966 stored in memory 956, as well as stores and retrieves application data. The processor 954 is included to be representative of a single processor, multiple processors, a single processor having multiple processing cores, and the like. The network interface 960 may be any type of network communications allowing the computing system 952 to communicate with computing system 902 via network 905.

The storage may 958 may be a disk drive storage device. Although shown as a single unit, the storage 958 may be a combination of a fixed and/or removable storage devices, such as fixed disk drives, removable memory cards, optical storage, network attached storage (NAS), or storage area network (SAN). As illustrated, the storage 958 may include reference colors 974 and biomarker levels 976. The reference colors 974 may include those reference colors that are provided by the manufacturer to which the computing environment 900 will compare the color of the captured image. For example, the reference colors 974 may include a separate color directed to “depleted” (poor biomarker level), then “low”, then “threshold” (adequate biomarker level), then “target” and finally “high”. The biomarker levels 976 include those readings to which each color represents. For example, the biomarker levels 976 include the correlations between a single color and a single level.

In the embodiment, the memory 956 includes an operating system 964, program code 966, website 968, color identifier 970, and biomarker level correlation agent 972. The website 968 is accessed by the computing system 902. The website 968 may correspond to a user's personal webpage that is managed by the web client application server. The program code 966 may be accessed by the processor 964 for processing. The program code 962 may include the steps discussed herein in conjunction with FIGS. 3, 5, 8, and 10 that are performed by the web client application server. The color identifier 970 is configured to detect the color that is indicated by the test pad 103 in the captured image. The biomarker level correlation agent 972 is configured to correlate the identified activation color with the biomarker level.

Although memory 956 is shown as a single entity, memory 956 may include one or more memory devices having blocks of memory associated with physical addresses, such as random access memory (RAM), read only memory (ROM), flash memory, or other types of volatile and/or non-volatile memory.

The computing environment 900 may further include one or more external devices 150. The one or more external devices 150 are configured to measure and record a health metric associated with the user. For example, the one or more external devices 150 may include wearable device(s) 152 and smart device(s) 154. An example wearable device 152 may include a smart watch that may be configured to track daily steps, calories burned, heart rate, sleep cycle, and the like. An example smart device 154 may be a wireless blood pressure device, a wireless weight scale, a wireless body fat monitor, and the like. The one or more external devices 150 are discussed in more detail below in conjunction with FIG. 10.

FIG. 10 is a flow diagram of a method 1000 of receiving health information from a user, according to one embodiment. As shown in FIGS. 1, 6, 7, and 9, the computing environment may also include one or more external devices 150.

At step 1002, the system receives a health input. In one example, the user device receives a health input directly from the user in the form of a manual entry by the user. In another example, the user device receives a health input directly from an external device 150 that is “tethered” or connected thereto. In another example, the web client application server receives a health input directly from an external device 150 that is in communication with the web client application server over a network.

At step 1004, the system updates the user's account with the received health input. In the example in which the client device receives the health input, the client device communicates with the web client application server to update the user's account with the received health input. In the example in which the web client application server directly receives the health input, the web client application server updates the user's account with the received health input.

Such input of additional health factors aids in allowing the web client application server to analyze incoming biomarker readings contextually. For example, such input of additional health factors allows the web client application server to analyze biomarker levels in the context of exercise type and food consumption. Such complete information aids in providing predictive feedback to the user.

For example, the system (e.g., the web client application server) may assess vascular wellness and fitness as defined as the ability to maintain a normal blood pressure range, lower blood pressure, extend time-to-exhaustion, reduce the need for oxygen consumption, improve cellular respiration, and/or mitochondrial efficiency through a combination of daily physical exercise. The daily physical exercise may include anaerobic or aerobic training. In another example, such factors may be improved through a diet consisting of leafy green vegetables, such as a Mediterranean diet, a DASH (dietary approach to stop hypertension) diet, or through any other suitable methods.

The method 1000 may optionally include steps 1006 and 1008. At step 1006, the web client application server may generate a message in response to the receive health input. For example, the message may be directed to the received heart rate level, the received blood pressure level, the received sleep cycle reading, and the like. The web client application server transmits the message to the client device for display. The client device then pushes the message to the user (step 1008). For example, the client device updates the GUI displayed on the screen to notify the user of the message.

As such, the present disclosure provides a self-testing biomarker method that leverages a mobile device platform that can accurately and automatically capture levels of real-time biomarkers and monitor such biomarkers in the context of daily activities to promote and maintain a desired state of wellness. The system may be used as part of a behavioral modification program for dietary control, heart healthy food consumption, or general fitness.

The current disclosure takes a data-driven, social networking approach to disease prevention that allows users/members to self-measure wellness, make lifestyle dietary adjustments, and then share “best practices” and outcomes with like-minded individuals. While individuals interact to help improve their outcomes, the data streams provided serve as the building blocks for a consumer-driven model “to eat smart, live well, and be fit” by self-testing and self-tracking, with the ability to make real-time adjustments.

The current disclosure aids in shifting society's current practice of managing chronic disease to preventing disease by providing a family of affordable, do-it-yourself, self-administered bodily fluid tests to monitor the levels of natural metabolites or “health biomarkers’ that every person's body makes to maintain health and wellness.

While the present disclosure has been discussed in terms of certain embodiments, it should be appreciated that the present disclosure is not so limited. The embodiments are explained herein by way of example, and there are numerous modifications, variations and other embodiments that may be employed that would still be within the scope of the present disclosure.

What is claimed:

1. A method of monitoring a level of a biomarker found in a bodily fluid of a user using a client device, comprising:
receiving an indication to capture a biomarker level reading, wherein the biomarker level reading is of a test pad on a test strip, the test pad containing a reactant disposed thereon that, when placed into contact with a sample of the bodily fluid, displays a color related to a level of the biomarker in the bodily fluid;
identifying, with a camera, a portion of the test strip containing the test pad displaying the color related to the level of the biomarker;
identifying the color displayed on the test pad;
correlating the identified color displayed on the test strip to the level of the biomarker; and
updating an account of the user with the determined biomarker level.

2. The method of claim 1, wherein identifying, with a camera, a portion of the test strip containing the test pad displaying the color related to the level of the biomarker, comprises:
overlaying a target on a screen of the client device;
automatically adjusting camera settings to identify a portion of the test strip;
identifying one or more features of test strip within a line of sight of the camera;
computing a score based on one or more of the identified features of the test;
upon determining that the computed score is greater than or equal to the threshold score, instructing the camera to capture the portion of the test strip within the line of sight of the camera.

3. The method of claim 1, wherein identifying the color displayed on the test strip, comprises:
identifying red-green-blue (RGB) colors displayed on the test strip within the target;
identifying a centrally occurring RGB color of the test strip;
converting the centrally occurring RGB color to a corresponding color in the LAB color space;
computing a distance between the corresponding color in the LAB color space and each reference color;
identifying the reference color with a smallest distance from the corresponding color in the LAB color space;
associate the color displayed on the test strip with identified reference color.

4. The method of claim 1, further comprising:
analyzing the determined biomarker level in context with one or more of exercise data, nutrition data, and health-related data.

5. The method of claim 1, further comprising:
receiving health-related information from an external wearable device, wherein the external wearable device is configured to monitor a bodily function of the user; and
analyze the determined biomarker level in conjunction with the received health-related information.

6. The method of claim 1, further comprising:
comparing the determined biomarker level to previously recorded biomarker levels; and
generating a notification for the user based on the comparison between the determined biomarker level and the previously recorded biomarker levels.

7. The method of claim 1, wherein identifying, with a camera, a portion of the test strip containing the test pad displaying the color related to the level of the biomarker, comprises:
capturing an image of the portion of the test strip with the camera; and
transmitting the image to a remote server for further analysis.

8. The method of claim 1, wherein identifying the color displayed on the test strip, comprises:
generating a training data set that includes at least one or more known biomarker concentration levels, an associated test pad display color for each of the one or more known biomarker concentration levels, and metadata associated with the camera of client device that captured each associated test pad display color; and
generating a prediction algorithm based on the training data set.

9. The method of claim 8, further comprising:
applying the prediction algorithm to color displayed on the test strip to predict a biomarker concentration level displayed on the test strip.

10. The method of claim 8, further comprising:
receiving user feedback relating to an accuracy of the prediction algorithm to further refine the prediction algorithm.

11. A system, comprising:
a processor; and
memory having instructions stored thereon, which, when executed by the processor, performs an operation of monitoring a level of a biomarker found in a bodily fluid of a user, comprising:
receiving an indication to capture a biomarker level reading, wherein the biomarker level reading is of a test pad on a test strip, the test pad containing a reactant disposed thereon that, when placed into contact with a sample of the bodily fluid, displays a color related to a level of the biomarker in the bodily fluid;
identifying, with a camera in communication with the system, a portion of the test strip containing the test pad displaying the color related to the level of the biomarker;
identifying the color displayed on the test strip;
correlating the identified color displayed on the test strip to the level of the biomarker; and
updating an account of the user with the determined biomarker level.

12. The system of claim 8, wherein identifying, with a camera in communication with the system, a portion of the test strip containing the test pad displaying the color related to the level of the biomarker, comprises:
overlaying a target on a screen of the client device;
automatically adjusting camera settings to identify a portion of the test strip;
identifying one or more features of test strip within a line of sight of the camera;
computing a score based on one or more of the identified features of the test;
upon determining that the computed score is greater than or equal to the threshold score, instructing the camera to capture the portion of the test strip within the line of sight of the camera.

13. The system of claim 11, wherein identifying the color displayed on the test strip, comprises:
identifying red-green-blue (RGB) colors displayed on the test strip within the target;
identifying a centrally occurring RGB color of the test strip;
converting the centrally occurring RGB color to a corresponding color in the LAB color space;
computing a distance between the corresponding color in the LAB color space and each reference color;
identifying the reference color with a smallest distance from the corresponding color in the LAB color space;
associate the color displayed on the test strip with identified reference color.

14. The system of claim 11, further comprising:
analyzing the determined biomarker level in context with one or more of exercise data, nutrition data, and health-related data.

15. The system of claim 11, further comprising:
receiving health-related information from an external wearable device, wherein the external wearable device is configured to monitor a bodily function of the user; and
analyze the determined biomarker level in conjunction with the received health-related information.

16. The system of claim 11, further comprising:
comparing the determined biomarker level to previously recorded biomarker levels; and
generating a notification for the user based on the comparison between the determined biomarker level and the previously recorded biomarker levels.

17. The system of claim 11, wherein identifying, with a camera, a portion of the test strip containing the test pad displaying the color related to the level of the biomarker, comprises:
capturing an image of the portion of the test strip with the camera; and
transmitting the image to a remote server for further analysis.

18. The system of claim 11, wherein identifying the color displayed on the test strip, comprises:
generating a training data set that includes at least one or more known biomarker concentration levels, an associated test pad display color for each of the one or more known biomarker concentration levels, and metadata associated with the camera of client device that captured each associated test pad display color;
generating a prediction algorithm based on the training data set; and
applying the prediction algorithm to color displayed on the test strip to predict the color displayed on the test strip.

19. The system of claim 18, further comprising:
receiving user feedback relating to an accuracy of the prediction algorithm to further refine the prediction algorithm.

20. A non-transitory computer readable medium having instructions store thereon, which, when executed by a processor, cause the processor to perform a method of generating a thumbnail for a media file, comprising:
receiving an indication to capture a biomarker level reading, wherein the biomarker level reading is of a test pad on a test strip, the test pad containing a reactant disposed thereon that, when placed into contact with a sample of the bodily fluid, displays a color related to a level of the biomarker in the bodily fluid;
identifying, with a camera, a portion of the test strip containing the test pad displaying the color related to the level of the biomarker;
identifying the color displayed on the test strip;
correlating the identified color displayed on the test strip to the level of the biomarker; and
updating an account of the user with the determined biomarker level.
